(19) United States
(12) Patent Application Publication
(10) Pub. No.: US 2005/0273063 A1
(43) Pub. Date: Dec. 8, 2005

(54) Surgical suction instrument

(76) Inventors: Thomas Hoell, Gutenberg (DE); Guido Fehling, Karlstei (DE)

Correspondence Address: PENDORF & CUTLIFF, 5111 Memorial Highway, Tampa, FL, 33634-7356, US

(21) Appl. No.: 11/120,416

(22) Filed: May 3, 2005

(30) Foreign Application Priority Data
May 3, 2004 (DE) 10 2004 021 676.2

(51) Int. Cl. A61M 1/00

(52) U.S. Cl. 604/317

(57) ABSTRACT

A suction instrument for surgery, wherein the grip and the suction pipe of the suction instrument are produced as separate parts. The grip is comprised of a handpiece with an axially extending inner channel. The suction pipe is introduced into the distal end of the hand piece and can thus be adjusted and varied in its effective working length by inserting the suction pipe different axially distances into the hand piece. In its respective adjusted axial position and rotation the suction pipe is force-fittingly fixed or friction locked in the hand piece. Friction locking allows the axial position of the suction pipe in the hand piece to be freely varied, so that an effective length of the suction pipe and its rotation position in regard to the hand piece can be adapted optimally to the respective operation requirements, and can be readjusted as the operation progresses. The suction pipe is sealed at its outer circumference in the hand piece, so that the vacuum, which is provided by the hose connected to the proximal end of the hand piece, is transmitted to the suction pipe.

### BACKGROUND OF THE INVENTION

1. Field of the Invention

The invention concerns a suction instrument for surgery according to the pre-characterizing portion of Patent Claim 1.

2. Related Art of the Invention

In surgery, suction devices are an important instrument used to suck blood and other fluids out of the field of operation. For this, the suction device is connected to a vacuum system via a flexible hose. The suction instrument includes a form-stable suction pipe, which terminates in a distal tip. A grip or handle is formed at the proximal end of the suction pipe, in order to hold the suction instrument and directe it in the field of operation. The length and the diameter of the suction pipe as well as the shape of the tip are adapted to the respective application purpose. The length can be between 5 cm and 60 cm. The diameter can be up to 30 mm. In neurosurgical operations, in particular in the brain, when working under an operation microscope, very fine suction pipes having a diameter as small as 1.5 mm are employed.

Further, in neurosurgery suction instruments are employed which have a vent opening in the area of the grip, which is closed by the thumb during the suction process. If the thumb is lifted from this vent opening, the vacuum instrument draws in air through this vent opening and the suction effect at the tip is accordingly reduced. If tissue is suctioned in undesired manner to the suction instrument tip during the operation, then a release of the vent at the side results in reduction of the vacuum at the suction tip so that the suctioned tissue is again released. This is important for example when operating in the vicinity of brain nerves and tissue, which are very sensitive and can be damaged or pulled out by the suction from the suction tip.

During the operation the operator lays his hand upon a fixed substrate in the immediate vicinity of the operation cavity, in order to avoid shaking of the hand and fatigue. As the surgical intervention advances deeper, as is generally the case for example when operating on the head, the operator increasingly requires various lengths of suction pipe, since the distance between the hand and the deepest location in the field of operation, at which the suction tip is to be employed, increases. Thus, With the conventional surgical suction instruments, suction instruments of different lengths and various diameters are laid out on the operating table, which the operator employs respectively depending upon the progress of the operation. Since the number of the various suction instruments is limited due to economical and practical reasons, it repeatedly occurs that operational conditions and geometries occur in which the operator must work with an inappropriate suction lengths.

### SUMMARY OF THE INVENTION

The invention is concerned with the task of alleviating this problem and to make available a suction instrument for accessing locations, which instrument allows an adaptation of the length of the suction pipe to the operational requirements.

This task is inventively solved by a suction instrument having the characteristics of Claim 1.

Advantageous embodiments and further developments of the invention are set forth in the dependent claims.

In accordance with the invention the grip and the suction pipe of the suction instrument are produced as separate parts. The grip is comprised of a handpiece with an axially extending inner channel. The suction pipe is introduced into the distal end of the hand piece and can thus be adjusted and varied in its effective working length by inserting the suction pipe different axially distances into the hand piece. In its respective adjusted axial position and rotation the suction pipe is force-fittingly fixed or friction locked in the hand piece. Friction locking allows the axial position of the suction pipe in the hand piece to be freely varied, so that an effective length of the suction pipe and its rotation position in regard to the hand piece can be adapted optimally to the respective operation requirements, and can be readjusted as the operation progresses. The suction pipe is sealed at its outer circumference in the hand piece, so that the vacuum, which is provided by the hose connected to the proximal end of the hand piece, is transmitted to the suction pipe.

In an advantageous embodiment the sealing of the suction pipe in the hand piece is accomplished by a seal, which is in the form of a hollow cylindrical elastic bushing received in the distal end of the hand piece. This seal simultaneously serves for the friction locking of the suction pipe in its respective axial position. Preferably the elastic bushing forming the seal can be tightened axially by a cap nut screwed onto the distal end of the hand piece, so that its inner diameter constricts and a stable axial and rotational fixing of the suction pipe results.

In a different embodiment, a chuck is formed on the distal end of the hand piece, which is pressed against the suction pipe by an inner cone of the screwed on cap nut. Thereby an axial fixing of the suction pipe in the hand piece is accomplished, which is even more stable against tilting of the axis of the suction pipe.

Preferably, the inner diameter of the inner channel of the hand piece is widened at least in the proximal area relative to the outer diameter of the suction pipe, so that an air gap remains in the inside of the hand piece. A vent closeable by the finger of the operator leads radially into this air gap, so that ventilation is made possible when the suction effect at the suction tip is to be reduced.

The two-part embodiment of the grip and the suction pipe makes possible an economical design and use of the suction instrument. Multiple suction pipes can be used interchangeably with the same hand piece. For example, used and in certain cases damaged suction pipes can be replaced with new and sterile suction pipes. Suction pipes with variously designed suction tips can be employed. Also, suction pipes with various diameters can be employed, wherein small differences of the outer diameter of the suction pipe can be accommodated by the axial constriction of the seal. In the case of greater differences of the outer diameter, seals with correspondingly different inner diameters can be employed. A color coding of the seals and the suction pipes would facilitate a simple and reliable coordination. The seal is preferably in the form of an axially extending bushing, which surrounds the suction pipe in the manner of a hose. The seal thereby supports the suction pipe against a tilting of the axis in the hand piece, which is important in particular when the suction pipe has radial free play in the inner channel of the hand piece.

The hand piece can be economically manufactured of plastic. A manufacturing from metal, in particular stainless steel, is possible, which is advantageous in particular for a reusable sterilizeable hand piece.

The suction pipe can likewise be produced economically from plastic. For many application purposes it is advantageous to produce the suction pipe from a deformable metal, so that the suction pipe can be bent during the operation into a useful shape. Since the required length of the suction pipe increases during the course of the operation, as a rule, during the extension of the suction pipe unbent suction pipe sections there are respectively pulled out of the hand piece, so that a rebending for adaptation to the progress of the operation is possible. Sealing always occurs in a not yet deformed axial area of the suction pipe. Herein the suction pipe can also be produced from a memory alloy so that suction pipes bent during the progress of the operation resume their original undeformed shape during subsequent heat sterilization.

For the connection of the suction instrument to the vacuum system, in general a hose is employed, which is semi-rigid, in order to resist the vacuum. The stiffness of the hose has the consequence that the inner lumen of the hose is aligned with, and axially extends, the inner channel of the hand piece. The suction pipe could thus extend with its proximal end beyond the proximal end of the hand piece and be inserted into the hose. Thereby a great variability of length of the suction pipe is possible, without the hand piece having to exhibit a correspondingly large axial length.

The economic advantage of the invention is comprised therein, that it is not necessary to maintain a large number of various suction instruments available on the operating table. It is sufficient to have one hand piece or a few hand pieces for the required number of suction pipes. Therein also the number of the suction pipes can be reduced, since their length is adjustable. The number of the required expensive hand pieces is significantly reduced, since the comparatively economical suction pipes can be employed in large numbers. For hygienic reasons it is advantageous, that the inexpensive suction pipes can be used as a single use disposable article.

Finally, the step-less axial length adjustment of the suction pipe improves the use of the suction instrument for the requirements of the operator.

### BRIEF DESCRIPTION OF THE DRAWINGS

In the following the invention is described in greater detail on the basis of an illustrative embodiment shown in the Figures. There is shown

FIG. 1 an axial section through a suction instrument,

FIG. 2 a top view upon the ventilation opening of the suction instrument,

FIG. 3 an axial section through a suction instrument in a second embodiment, and

FIG. 4 a cross section according to Lines A-A in FIG. 3

### DETAILED DESCRIPTION OF THE INVENTION

The suction instrument includes a grip, comprised of a hand piece 10. The hand piece 10 has the basic shape of a pipe and is ergonomically optimized for guidance by the hand of the operator. An inner channel 12 passes in the axial direction through the hand piece 10, which preferably exhibits a circular cross section. At the proximal end of the hand piece 10 there are connecting means 14 for securing a hose for the supply of the vacuum. This connecting means could be a tube clip, screw connection or be otherwise variously designed.

A shape-stable suction pipe 16 can be introduced with its proximal end from the distal side to the inner canal 12 of the hand piece 10. The suction pipe 16 has an outer diameter, which is slightly smaller than the inner diameter of the inner channel 12, so that the vacuum hose 16 is axially slideable in the hand piece 10 and is substantially supported against radial tilting. At the distal end the suction pipe can be shaped to form a suction tip 18.

At the distal end of the hand piece 10 the inner channel 12 is widened in its diameter to a receptacle 24. In this receptacle 24 a seal 20 is seated in the embodiment according to FIG. 1. The seal 20 has the shape of an elongated hollow cylindrical bushing and is comprised of an elastic deformable plastic material. The outer diameter of the seal 20 corresponds to the inner diameter of the receptacle 24, while the inner diameter of the seal 20 corresponds to the outer diameter of the suction pipe 16. The seal 20 is supported with its proximal end axially in an inner shoulder, which is formed by the proximal end of the receptacle 24. The distal end of the seal 20 projects somewhat beyond the distal end of the hand piece 10. A cap nut 22 can be screwed onto an outer threading at the distal end of the hand piece 10, through which the suction pipe 16 passes freely.

If the cup nut 22 is loosened, that is, screwed in the distal direction, then the seal 20 is relaxed. The suction pipe 16 can thus be displaced axially in the seal 20, and therewith in the inner channel of the hand piece 10, at which time the seal 20 causes only a slight friction. If the suction pipe 16 is positioned in the desired axial position in the hand piece 10, then the cap nut 22 is screwed on in the proximal direction onto the hand piece 10. At this time the cap nut 22 presses on the distal projecting end of the seal 20 and compresses the seal 20 axially together in the receptacle 24. Thereby the wall thickness of the seal 20 expands and the seal 20 lies sealingly and with radial pressure against the inner wall of the receptacle 24 and against the outer circumference of the suction pipe 16. In this manner this seal 20 seals the suction pipe 16 air tight in the hand piece 10 and fixes the suction pipe 16 by friction locking against axial displacement and rotation in the hand piece 10.

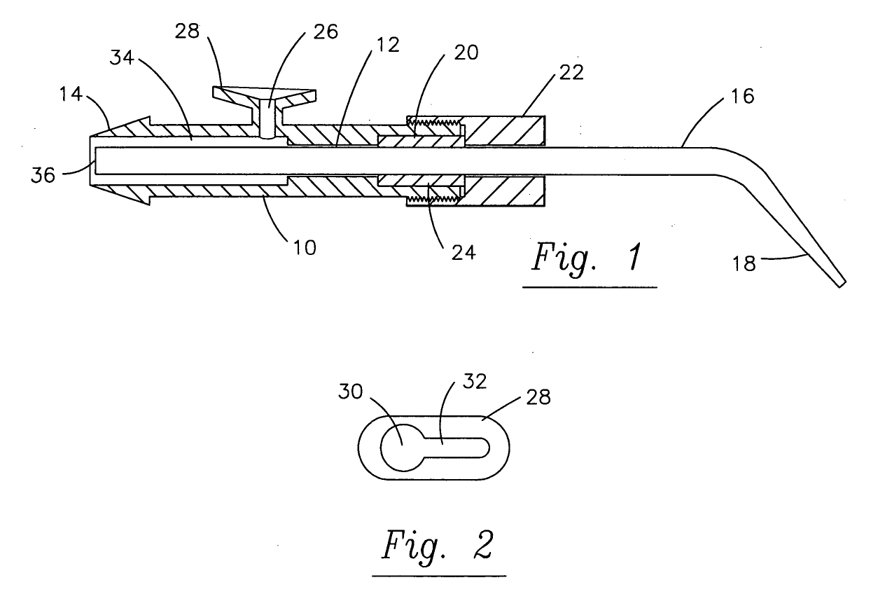

Proximally behind the seal 20 and spaced apart axially therefrom the wall of the hand piece 10 is interrupted by a vent opening 26. On the outer side of the hand piece 10 the exit end of the vent opening 26 is surrounded by a synclinal finger plate 28. The cross section of the ventilation opening 26 has, as shown in FIG. 2, the shape of a key hole, with a circular section 30 of enlarged diameter at the proximal end, and an elongated narrow section 32 which extends in the distal direction. The ventilation opening 26 communicates in an axial air gap 34 between the inner wall of the inner channel 12 and, as the case may be, the inserted suction pipe 16, which is in communication via the connection means 14 with the hose and to therewith the vacuum source. The air gap 34 can be in the form of an axial groove, which extends in the inner wall of the inner channel 12 from the proximal end to the ventilation opening 26. Preferably, however, the diameter of the inner channel 12 from the proximal end to the ventilation opening 26 is widened, so that an air gap 34 remains free in the form of a ring gap between the inner wall of the inner channel 12 and the vacuum pipe 16. The operator closes, with his thumb seated upon the finger plate 28, the air opening 26 so that the vacuum is effective from the vacuum pipe 16 to the suction pipe 18. If the operator lightly lifts his thumb from the finger plate 28, then he first frees a narrow section 32 of the ventilation opening 26. By the small breadth of the narrow section 32 the operator can thus allow entry of finely controlled restricted air through the ventilation opening 26, in order to reduce the suction effect at the suction tip 18 with good fingertip control. If the operator completely lifts his thumb from the finger plate 28 then he also exposes the round section 30, so that air can enter via a larger cross section of the ventilation opening 26 and so that no effective suction occurs any longer at the suction tip 18.

If the suction pipe 16 is a reusable suction pipe 16 of metal, then the proximal end 36 of the suction pipe 16 can be introduced into a rinse connection, for example a Luer-lock- connection, so that the vacuum pipe 16 can be flushed for cleaning.

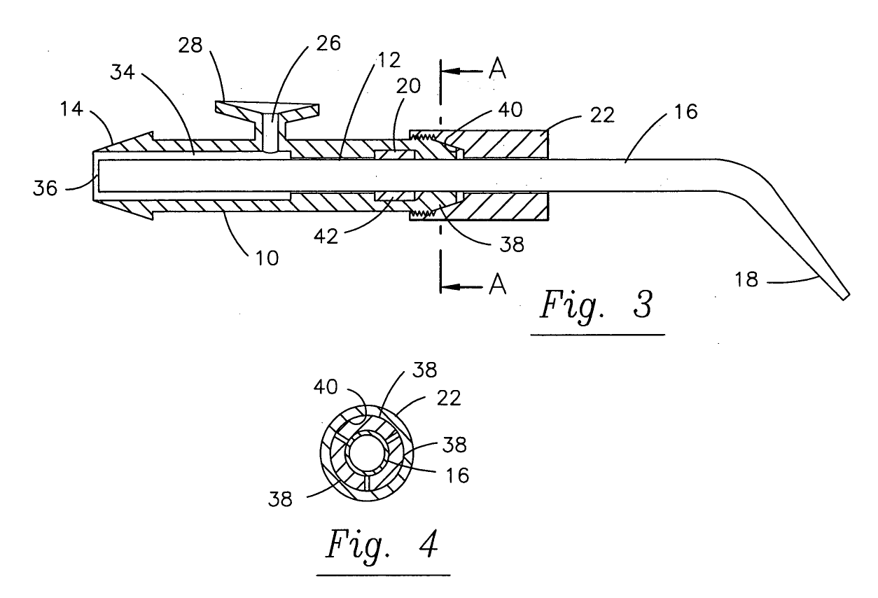

In an embodiment shown in FIGS. 3 and 4 the distal end of the hand piece 10 is in the form of a chuck with three or four elastic clamp jaws 38. The clamp jaws 38 have an outer cone, and are pressed via an inner cone 40 of the cap nut 22 against the outer circumference of the suction pipe 16, when the cap nut 22 is screwed onto the hand piece 10. Thereby the suction pipe 10 is clamped with high stability in the hand piece 10 and fixed against axial displacement and rotation. For sealing, a sealing ring 42 can be introduced in the ring gap between the suction pipe 16 and the hand piece 10, as shown in FIG. 3. Alternatively a seal can also be introduced on the suction pipe 16 distally in front of the cap nut.

### Reference Number List

10 Hand piece
12 Inner channel
14 Connection means
16 Suction pipe
18 Suction tip
20 Seal
22 Cap Nut
24 Receptacle
26 Ventilation opening
28 Finger plate
30 Round section
32 Narrow section
34 Air gap
36 Irrigation connection
38 Clamp jaw
40 Inner cone
42 Sealing ring

1. A suction instrument for surgery, with
a grip,
a shape stable suction pipe provided distally on the grip and
connection means for a hose provided proximally on the grip,
wherein the grip is comprised of a hand piece (10) separate from the suction pipe (16),
wherein the hand piece includes an inner channel (12) extending axially therethrough, and
wherein the suction pipe (16) is adapted to be introduced axially displaceable, fixable by friction locking and sealed into the distal end of the hand piece (10), such that the axial length to which the suction pipe (16) extends into or, as the case may be, projects from the inner channel (12) is adjustable.

2. The suction instrument according to claim 1, wherein a seal (20) is introduceable in the distal end of the hand piece (10), which sealingly closes the ring gap between the inner channel (12) and the suction pipe (16) and friction locks the suction pipe (16) in the hand piece (10).

3. The suction instrument according to claim 2, wherein the seal (20) is a hollow cylindrical elastic bushing, which is introduced in the receptacle (24) of the inner channel (12) and is held and axially compressed by a cap nut (22) threadably seated upon the distal end of the hand piece (10).

4. The suction instrument according to claim 1, wherein a chuck is formed on the distal end of the hand piece (10), which is pressed against the suction pipe (16) by a cap nut (22) screwed upon the hand piece (10).

5. The suction instrument according to claim 1 wherein the inner channel (12) forms in the proximal end area a air gap (34) open to the proximal end and that a vent opening (26) extends through the wall of the hand piece (10) in this air gap (34).

6. The suction instrument according to claim 5, wherein the vent opening (26) is provided on its outer exit end with a finger plate (28).

7. The suction instrument according to claim 6, wherein the vent opening (26) exhibits a key hole shaped cross section, of which the narrower section (32) is directed distally.

8. The suction instrument according to claim 1, wherein the suction pipe (16) is of plastic.

9. A suction instrument according to claim 1, wherein the suction pipe (16) is metal.

10. The suction instrument according to claim 9, wherein the suction pipe (16) is comprised of a deformable metal.

11. The suction pipe instrument according to claim 10, wherein the suction pipe (16) is comprised of a memory alloy.

12. The suction instrument according claim 9 wherein the proximal end of the suction pipe (16) is provided with an irrigation or rinsing connection (36), in particular a Luer-lock-connection.
